(19) United States
(12) Patent Application Publication
(10) Pub. No.: US 2021/0298631 A1
(43) Pub. Date: Sep. 30, 2021

(54) SYSTEM AND METHOD FOR MONITORING IMPLANT FIXATION USING ELECTRICAL IMPEDANCE TOMOGRAPHY

(71) Applicant: THE GENERAL HOSPITAL CORPORATION, Boston, MA (US)

(72) Inventors: Kartik Mangudi Varadarajan, Acton, MA (US); Tyler Tallman, West Lafayette, IN (US); A. John Hart, Waban, MA (US)

(21) Appl. No.: 17/269,763

(22) PCT Filed: Aug. 22, 2019
(86) PCT No.: PCT/US19/47710

§ 371 Date: Feb. 19, 2021

Related U.S. Application Data
(60) Provisional application No. 62/721,640, filed on Aug. 23, 2018.
(60) Provisional application No. 62/722,127, filed on Aug. 23, 2018.

(51) Int. Cl. A61B 5/0536; A61B 5/00

(52) U.S. Cl. CPC A61B 5/0536; A61B 5/4851; A61F 2/36; A61B 2505/05; A61B 5/742

(57) ABSTRACT

A system for determining conditions within or near an implant apparatus in region of a subject includes an implant apparatus configured to be piezoresistive, the implant apparatus positioned in a region of a subject having a surface, and an electrical impedance tomography system. The electrical impedance tomography system includes a plurality of electrodes disposed on the surface of the region of the subject, each of the plurality of electrodes configured to apply a current to the region of the subject and to receive measurement signals from the region of the subject in response to an applied current. The electrical impedance tomography system further includes a current source coupled to the plurality of electrodes and configured to provide a current to at least one of the plurality of electrodes and a controller coupled to the plurality of electrodes and the current source, the controller configured to control the current source to provide a current to at least one of the plurality of electrodes, to receive the measurement signals from at least one of the plurality of electrodes, and to determine at least one electrical property associated with the implant apparatus based on the measurement signals. The electrical impedance tomography system also includes a display coupled to the controller and configured to display the at least one electrical property.

### CROSS-REFERENCE TO RELATED APPLICATIONS

This application is based on, claims priority to, and incorporates herein by reference in its entirety U.S. Ser. No. 62/721,640 filed Aug. 23, 2018, and entitled “Monitoring of Implant Fixation Using Electrical Impedance Tomography” and U.S. Ser. No. 62/722,127 filed Aug. 23, 2018 and entitled “Monitoring of Implant Fixation Using Electrical Impedance Tomography.”

### FIELD

The present disclosure relates generally to joint implants and in particular to a system and method to evaluate implant performance including determining conditions at or near fixation interfaces of an implant apparatus in a subject to, for example, determine implant fixation relative to bone.

### BACKGROUND

Diseases of the human joint such as osteoarthritis affect a significant proportion of the adult population, with tremendous impact on quality of life and productivity. Joint replacement surgery is one of the most common orthopedic procedures, with over 2 million procedures performed annually. However, failure of the surgery can occur both in the short-term and long term, posing a significant risk to patient health and placing an enormous economic stress on the healthcare system. Failure of the joint replacement surgery may require revision surgery to replace the implant (or prosthesis).

One of the most common causes of revision surgery is the loss of implant fixation relative to the bone. Implant loosening or loss of fixation can occur in association with peri-prosthetic infection (septic loosening) or in the absence of infection (aseptic loosening). Aseptic loosening can occur due to a number of reasons including inflammatory response to particulate debris generated from the implant, bone remodeling due to stress-shielding, or bone remodeling related to the ageing process.

Regardless of the cause of the revision, determining whether an implant is loose inside the joint of a patient is challenging. The most common method of diagnosing a loose implant involves use of plain, two-dimensional (2D) radiographs. The radiographs are visualized to determine the location and extent of radiolucent zones around the implant, which may indicate loosening of the implant at these locations. Such radiolucent lines may be seen at the cement-bone or cement-implant interfaces in the case of a cemented prosthesis, and at the metal-bone interface in the case of a cementless prosthesis (which do not require bone-cement for fixation). However, the presence of a radiolucent zone alone is not sufficient to confirm a loose implant, as radiolucent lines are present even in well-fixed implants immediately following surgery. Nonetheless, if the radiolucent zone progressively increases in size and extent around the implant, the likelihood that the implant is loose, increases. For example, loosening may be strongly suspected if radiolucent zone progresses over time, exceeds 2 mm in width, and extends around significant portion of the implant. Thus, determining whether an implant is loose using standard radiographic methods is highly subjective, and often requires following the patient over time with periodic collection and analysis of radiographs. In addition to the relevance of radiolucent lines, other challenges with plain radiographs include sensitivity to imaging direction, underestimating extent of radiolucency (since out-of-plane radiolucent regions may not be visible or may be blocked by prosthesis), etc.

Three-dimensional radiographic imaging methods such as computed tomography (CT) can be more sensitive for detecting radiolucent lines. However, they are more expensive and expose patients to a higher dose of ionizing radiation. Other methods for diagnosing loose implants include arthrography, where contrast agents are injected into the joint to delineate areas of interest on plain radiographs; scintigraphy, where radioisotopes are used as radiation sources and gamma cameras are used to create 2D images; and fluorodeoxyglucose-positron emission tomography (FDG-PET), which uses radionuclide (FDG) to create 3D visualization of the transport and metabolic rate of glucose. These techniques can be combined with plain radiographs to increase the sensitivity and specificity of detecting implant loosening. However, they involve more invasive procedures with potential risk of infection or allergic reaction to contrast agent, and increased expense.

In addition to the limitation discussed above, current imaging techniques are primarily a means to detect when an implant is grossly or completely loose. The ability to study conditions (e.g., stress/strain, crack-growth, crack formation, interface failure etc.) within the implant, or at or near fixation interfaces (e.g., implant-bone, bone-cement or cement-implant interfaces) could enable a better understanding of how various patient, surgical, and implant design factors affect implant performance. Early detection of failures or the ability to predict failures could also reduce the risk of implanting a flawed device in a large patient population, and enable intervention before significant bone loss occurs, thus reducing complexity of revision surgery. The ability to better understand mechanical loads (e.g., stresses, strains and deformation) experienced by implants could also be valuable for developing better implants.

Therefore, there is a need for a system and method to study and evaluate implant performance post-surgery, including systems and methods to diagnose implant loosening and to study conditions at the fixation interfaces.

### SUMMARY

In accordance with an embodiment, a system for determining conditions within or near an implant apparatus in region of a subject includes an implant apparatus configured to be piezoresistive, the implant apparatus positioned in a region of a subject having a surface, and an electrical impedance tomography (EIT) system. The electrical impedance tomography system includes a plurality of electrodes disposed on the surface of the region of the subject, each of the plurality of electrodes configured to apply a current to the region of the subject and to receive measurement signals from the region of the subject in response to an applied current. The electrical impedance tomography system further includes a current source coupled to the plurality of electrodes and configured to provide a current to at least one of the plurality of electrodes and a controller coupled to the plurality of electrodes and the current source, the controller configured to control the current source to provide a current to at least one of the plurality of electrodes, to receive the measurement signals from at least one of the plurality of electrodes, and to determine at least one electrical property associated with the implant apparatus based on the measurement signals. The electrical impedance tomography system also includes a display coupled to the controller and configured to display the at least one electrical property.

In accordance another embodiment, a method for determining conditions within or near an implant apparatus in region of a subject includes providing a plurality of electrodes on the surface of the region of the subject, the region of the subject having a surface and including an implant apparatus configured to be piezoresistive, applying a current to the region of the subject with at least one of the plurality of electrodes, detecting measurement signals with at least one of the plurality of electrodes, the measurement signals generated in response to the applied current, determining at least one electrical property associated with the implant apparatus based on the measurement signals, and displaying the at least one electrical property on a display.

### BRIEF DESCRIPTION OF THE DRAWINGS

The present disclosure will hereafter be described with reference to the accompanying drawings, wherein like reference numerals denote like elements.

FIG. 1 is a block diagram of a system for determining conditions within or near an implant apparatus in a subject in accordance with an embodiment;

FIG. 2 shows an exemplary femoral prosthesis, i.e., the femoral stem of a hip replacement prosthesis, in accordance with an embodiment;

FIG. 3 shows a transverse plane cross section (line a-a of FIG. 2) through a femur bone and femoral prosthesis fixed with bone cement in accordance with an embodiment;

FIG. 4 shows a transverse plane cross section (line a-a of FIG. 2) through a femur bone and femoral prosthesis without bone cement in accordance with an embodiment;

FIG. 5 shows a transverse plane cross section of an exemplary femoral prosthesis implant apparatus with composite bone cement in accordance with an embodiment;

FIG. 6 shows a transverse plane cross section of an exemplary femoral prosthesis implant apparatus formed with a composite material and fixed to a bone without the use of bone cement in accordance with an embodiment;

FIG. 7 shows a transverse plane cross section of an exemplary femoral prosthesis implant apparatus formed with a composite material and fixed to a bone with bone cement in accordance with an embodiment;

FIG. 8 shows a transverse plane cross section of an exemplary femoral prosthesis implant apparatus with a piezoresistive coating and fixed to a bone without the use of bone cement in accordance with an embodiment;

FIG. 9 shows a transverse plane cross section of an exemplary femoral prosthesis implant apparatus with a piezoresistive coating and fixed to a bone with bone cement in accordance with an embodiment;

FIG. 10 shows an example cuff having a plurality of electrodes in accordance with an embodiment;

FIG. 11 shows an example cuff having a plurality of electrodes on a mobile gantry that rotates in a circumferential direction in accordance with an embodiment;

FIG. 12 shows an example cuff having a plurality of electrodes on a mobile gantry that translates in a longitudinal direction in accordance with an embodiment;

FIG. 13 shows an exemplary femoral prosthesis, i.e., the femoral stem of a hip replacement prosthesis, and a cuff positioned around the joint in accordance with an embodiment;

FIG. 14 shows a transverse plane cross section of an exemplary femoral prosthesis implant apparatus fixed to a bone with bone cement with a portion of a bone-cement interface occupied by connective tissue in accordance with an embodiment;

FIG. 15 shows a transverse plane cross section of an exemplary femoral prosthesis implant apparatus fixed to a bone without the use of bone cement with a portion of a bone-implant interface occupied by connective tissue in accordance with an embodiment;

FIG. 16 shows an exemplary implant apparatus used to fix bone fractures and a cuff positioned around the joint in accordance with an embodiment;

FIG. 17 shows an example computational model of the human thigh;

FIG. 18 shows an example cross section computational model of a femoral prosthesis within a femur bone in accordance with an embodiment;

FIG. 19 shows an example computational model of the bone cement 260 of FIG. 17 with a simulated crack 262 along a length 264 of the bone cement 260;

FIG. 20A shows an example EIT image of the cross section along line b-b in FIG. 17 in the absence of a crack in the bone cement;

FIG. 20B shows an example EIT image of the cross section along line b-b in FIG. 17 in the presence of a longitudinal crack in the bone cement as shown in FIG. 18;

FIG. 21 shows an example computational model of the femoral prosthesis of FIG. 17 with an axial displacement of the femoral prosthesis within the joint;

FIG. 22A shows an example EIT image of the cross section along line b-b in FIG. 17 in the absence of displacement of the femoral prosthesis;

FIG. 22B shows an example EIT image of the cross section along line b-b in FIG. 17 in the presence of an axial displacement of the femoral prosthesis as shown in FIG. 20;

FIG. 23 shows an example computational model of an implant apparatus used to fix bone fractures and a cuff positioned around the joint;

FIG. 24 shows an example EIT image showing a change in conductivity within the fracture shown in FIG. 23 relative to an unloaded condition;

FIG. 25 shows an example experimental setup including an experimental test phantom;

FIG. 26 shows example EIT images illustrating conductivity changes of a bone cement with 1.0 vol. % carbon fiber as a function of applied load;

FIG. 27 shows example EIT images illustrating conductivity changes of a bone cement with 1.5 vol. % carbon fiber as a function of applied load;

FIG. 28 shows example EIT images illustrating conductivity changes of a bone cement with 2.0 vol. % carbon fiber as a function of applied load;

FIG. 29A illustrates an example finite element mesh used for EIT reconstructions;

FIG. 29B is an example graph showing average magnitude of conductivity change as imaged by EIT versus applied force;

FIG. 30 shows example EIT images obtained post failure for bone cement specimens with 1.0 vol. % CF, 1.5 vol. % CF and 2.0 vol. % CF;

FIG. 31 shows an example EIT image of a 1.5 vol. % CF specimen without any constraints on the conductivity change;

FIG. 32A shows an example specimen with a bone cement CF volume fraction of 1.0 vol. % CF post failure;

FIG. 32B shows an example specimen with a bone cement CF volume fraction of 1.5 vol. % CF post failure; and

FIG. 32C shows an example specimen with a bone cement CF volume fraction of 2.0 vol. % CF post failure.

### DETAILED DESCRIPTION

The present disclosure describes a system and methods for determining conditions within or near an implant (or prosthesis) in a subject (e.g., in a joint of a patient). The system and methods described herein may be used to, for example, study the performance of implants (e.g., orthopedic implants), study the environment in which the implant functions within a subject (e.g., inside a human body), evaluate the implant fixation relative to bone, and predict potential failure of an implant. The system includes an implant apparatus including, for example, piezoresistive bone cement, a piezoresistive implant, or a piezoresistive implant coating that experience changes in one or more electrical properties (e.g., impedance, conductivity, permittivity, or resistivity) in response to the loading environment and/or conditions at or near the implant apparatus or fixation interfaces (e.g., an implant-bone interface, an cement-bone interface, or a cement-implant interface). An electrical impedance tomography (EIT) system is used to measure the electrical properties or changes in the electrical properties. The measured electrical property or the measured change in an electrical property may be used to determine and evaluate the loading environment and/or conditions (e.g., stress/strain, crack formation, crack growth, implant motion, implant deformation, etc.) at or near the implant apparatus or the fixation interfaces. The system and methods described herein enable the non-invasive detection of implant performance without the use of ionizing radiation or injection of contrast agents. In addition, the system and methods may provide for a reduction in cost for diagnosing implant failure, such as implant loosening, which is the primary cause of revision surgery following joint replacement procedures.

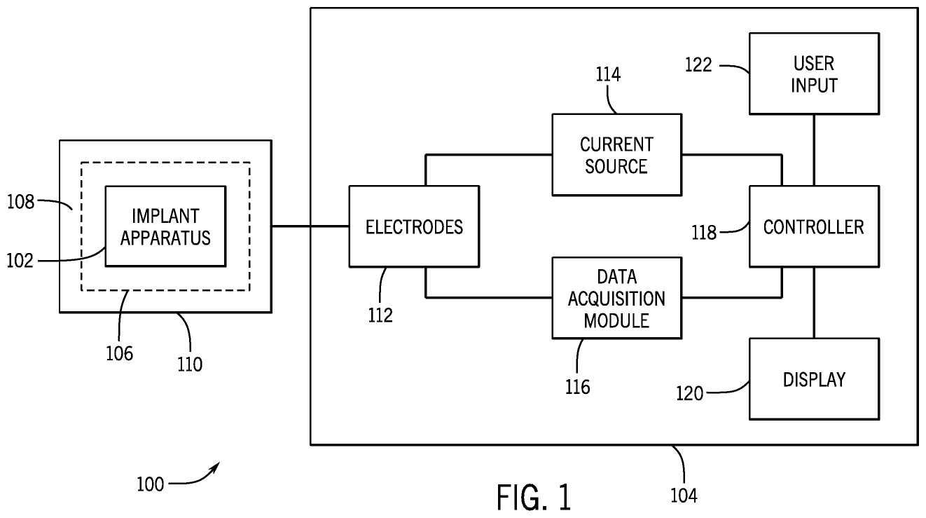

FIG. 1 is a block diagram of a system for determining conditions within or near an implant apparatus in a subject in accordance with an embodiment. System 100 includes an implant apparatus 102 and an electrical impedance tomography (EIT) system 104. Implant apparatus 102 is positioned in a region 108 of a subject, for example a joint in the body of a patient. Implant apparatus 102 may be, for example, any type of orthopedic implant such as an implant used to replace portions of a joint (e.g., knee, hip, spine, shoulder, ankle), an implant used to stabilize fractures (e.g., bone plates, screws, rods), or an implant used to fill and stabilize bone defects (e.g., mesh cages), etc. In one embodiment, the implant apparatus 102 may include bone cement 106 which is disposed around the implant in the region 108 to fix the implant within, for example, the bone. In another embodiment, the implant apparatus 102 may be fixed within the bone without bone cement, i.e., a cementless implant or prosthesis.

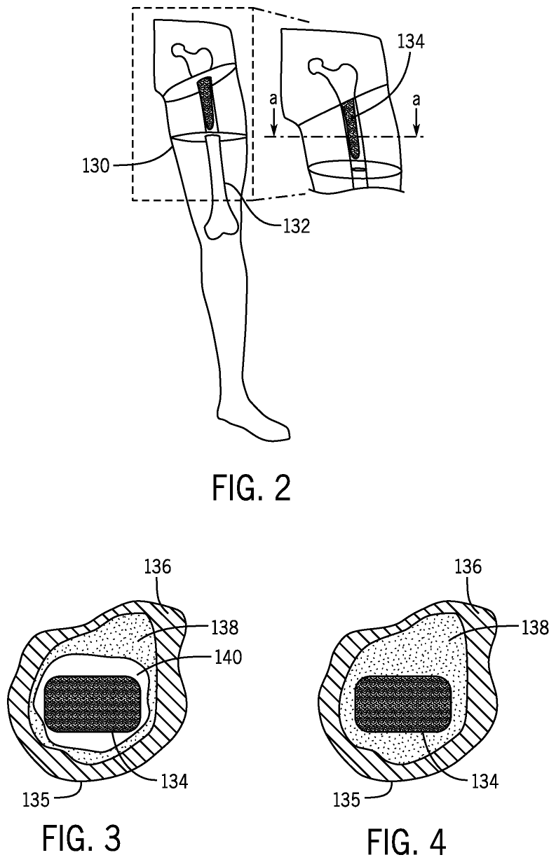

FIG. 2 shows an exemplary femoral prosthesis, i.e., the femoral stem of a hip replacement prosthesis, in accordance with an embodiment. In FIG. 2, a thigh 130 of the subject is shown including the femur bone 132. A femoral prosthesis 134 is implanted within the femur bone 132. As mentioned, an implant apparatus such as femoral prosthesis 134 may be fixed to bone, e.g., femur bone 132 using bone cement. FIG. 3 shows a transverse plane cross section (line a-a of FIG. 2) through a femur bone and femoral prosthesis fixed with bone cement in accordance with an embodiment. In FIG. 3, the cortical bone 136 and the trabecular bone 138 of the femur bone 135 are shown. The femoral prosthesis 134 (or implant) is fixed within the femur bone 135 using bone cement 140. In another embodiment, the implant may be a cementless implant. FIG. 4 shows a transverse plane cross section (line a-a of FIG. 2) through a femur bone and femoral prosthesis without bone cement in accordance with an embodiment. In FIG. 4, the cortical bone 136 and the trabecular bone 138 of the femur bone 135 are shown. The femoral prosthesis 134 (or implant) is fixed within the femur bone 135 without the use of bone cement (cementless implant). While the various embodiments described herein may refer to the femoral prosthesis shown in FIGS. 2-4 as an example implant apparatus 102, it should be understood that, as mentioned above, the implant apparatus may be any type of orthopedic implant.

Returning to FIG. 1, implant apparatus 102 (either with or without bone cement 106), is configured to be piezoresistive. In the various embodiments described below, the implant apparatus 102 may include piezoresistive bone cement, a piezoresistive implant, or a piezoresistive implant coating. As used herein, the term “piezoresistive” relates to a material whose electrical resistivity, conductivity, permittivity or impedance changes as a result of mechanical stress, mechanical strain, mechanical load, deformation, formation of cracks, growth of cracks, changes in temperature, etc. As used herein, the term “composite” refers to a material in which two or more distinct, for example, a “matrix” material and a “filler” material, are combined to provide structural or functional properties not present in an individual material.

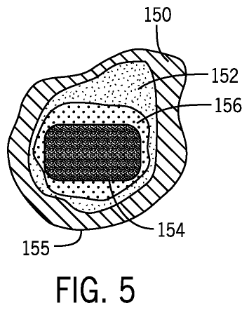

In one embodiment, the implant apparatus 102 includes bone cement that is formed as a composite containing conductive fillers. FIG. 5 shows a transverse plane cross section of an exemplary femoral prosthesis implant apparatus with composite bone cement in accordance with an embodiment. In FIG. 5, the cortical bone 150 and the trabecular bone 152 of a femur bone 155 are shown. A femoral prosthesis or implant 154 is positioned within the femur bone 155 and bone cement 156 is disposed around the implant 154 to fix the implant 154 to the femur bone 155. The bone cement 156 contains conductive fillers which give rise to piezoresistive behavior. Bone cement 156 may be any type of bone cement used for orthopedic applications including, for example, polymethyl methacrylate (PMMA). The conductive fillers may be any filler including one or more microscale and/or nanoscale fillers such as, for example, single-walled carbon nanotube (CNT), multi-walled CNTs (MWCNTs), graphene, nickel nanostrands, nickel powder, silver nanowires, gold nanowires, nickel-coated carbon fibers, etc.

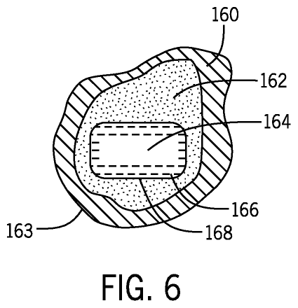

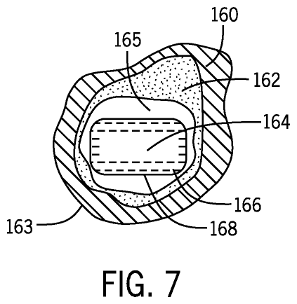

In another embodiment, the implant apparatus 102 includes an implant or a portion of the implant such as, for example, a bone interfacing portion of the implant, which is formed from a composite containing conductive fillers. FIG. 6 shows a transverse plane cross section of an exemplary femoral prosthesis implant apparatus formed with a composite material and fixed to a bone without the use of bone cement in accordance with an embodiment. FIG. 7 shows a transverse plane cross section of an exemplary femoral prosthesis implant apparatus formed with a composite material and fixed to a bone with bone cement in accordance with an embodiment. In FIGS. 6 and 7, the cortical bone 160 and the trabecular bone 162 of a femur bone 163 are shown. A femoral prosthesis or implant 164 is positioned within the femur bone 163. In FIG. 6, the implant 164 is a cementless implant. In FIG. 7, bone cement 165 is disposed around the implant 164 to fix the implant 154 to the femur bone 163. The implant 164 in FIGS. 6 and 7 is formed from a composite that contains conductive fillers 166 which give rise to piezoresistive behavior. In the embodiments shown in FIGS. 6 and 7, the conductive fillers 166 are dispersed near a bone interface portion 168 of the implant 164. The composite implant 164 may be composed of a polymer matrix such as PEEK (polyether ether ketone), PEKK (polyether ketone ketone), UHMWPE (ultra-high-molecular-weight polyethylene), etc., with the conductive filler 166 included within the polymer matrix. The conductive fillers 166 may be any filler including one or more microscale and/or nanoscale fillers such as, for example, single-walled (SW) CNT, MWCNTs, graphene, nickel nanostrands, nickel powder, silver nanowires, gold nanowires, nickel-coated carbon fibers, etc.

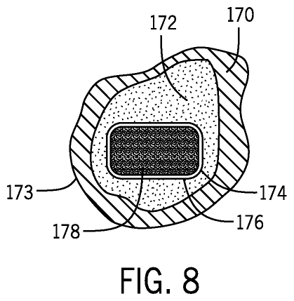

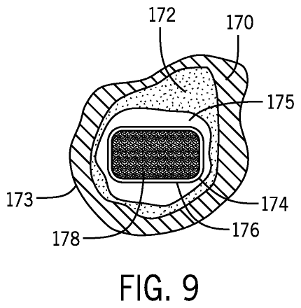

In yet another embodiment, the implant apparatus 102 includes an implant or a portion of the implant such as, for example, a bone interfacing portion of the implant, which is coated with a piezoresistive material. FIG. 8 shows a transverse plane cross section of an exemplary femoral prosthesis implant apparatus with a piezoresistive coating and fixed to a bone without the use of bone cement in accordance with an embodiment. FIG. 9 shows a transverse plane cross section of an exemplary femoral prosthesis implant apparatus with a piezoresistive coating and fixed to a bone with bone cement in accordance with an embodiment. In FIGS. 8 and 9, the cortical bone 170 and the trabecular bone 172 of a femur bone 173 are shown. A femoral prosthesis or implant 174 is positioned within the femur bone 173. In FIG. 8, the implant 174 is a cementless implant. In FIG. 9, bone cement 175 is disposed around the implant 174 to fix the implant 174 to the femur bone 173. A piezoresistive material or coating is applied to an implant bone interface 178 of the implant 174. The piezoresistive material may be, for example, conductive liquid silicone rubber, conductive nanosilver ink, conductive graphene ink, etc.

Returning to FIG. 1, the piezoresistive bone cement, piezoresistive implant or the piezoresistive implant coating of the implant apparatus 102 is configured to experience changes in one or more electrical properties, for example, impedance, conductivity, permittivity, or resistivity, in response to the loading environment and/or conditions at or near the fixation interfaces. EIT system 104 may be used to generate an image of one or more electrical properties (e.g., conductivity, permittivity, etc.) of the region 108 of the subject and the implant apparatus 102 positioned within the region 108 based on surface electrode measurements. EIT system 103 includes a plurality of electrodes 112, a current source 114, a data acquisition module 116, a controller 118, a display 120 and a user interface 122. The plurality of electrodes 112 are configured to be attached to a surface 110 of the region 108 of the subject to measure electrical properties or changes in electrical properties in the region 108 and/or the implant apparatus 102. In an embodiment, the plurality of electrodes 112 may be part of an external device that may be placed on or around the region 108 of the subject having the implant apparatus 102. For example, the region 108 may be a joint of a patient, the surface 110 is the skin of the patient, and the implant apparatus 102 may be a joint prosthesis. The external device with the plurality of electrodes 112 may be placed on the skin of the patient around the joint having the joint prosthesis. Electrical properties at a desired location, for example, a desired cross section, may be measured by operating a select number of the plurality of electrodes 112 as discussed further below. For example, a select number of the plurality of electrodes 112 at the level of the desired cross-section may be operated to measure electrical properties at the desired cross-section.

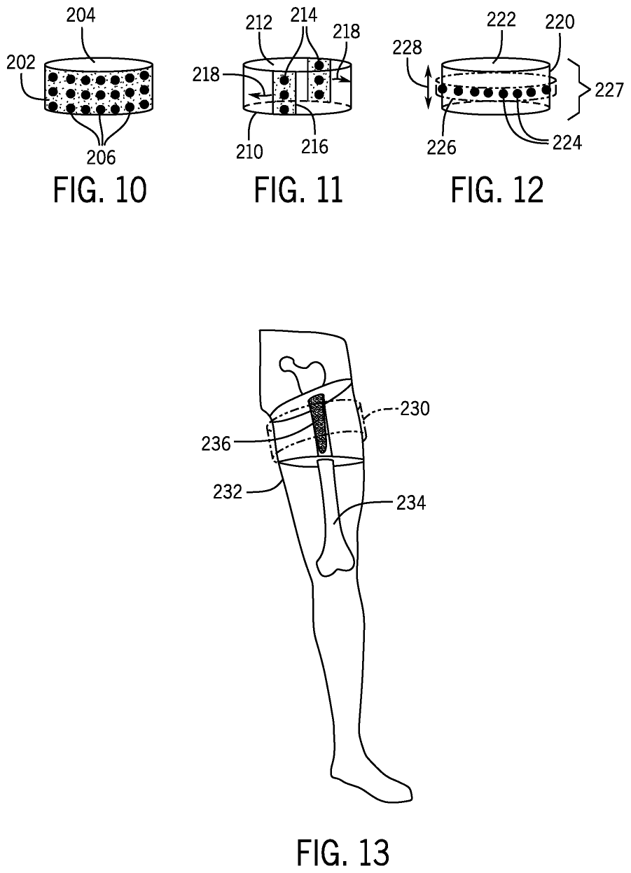

In one embodiment, the external device may be a cuff that is configured to be placed around the joint and the plurality of electrodes 112 are positioned on the cuff. The electrodes may be arranged in one or more of a circumferential, longitudinal or other direction. FIGS. 10-12 show various embodiments of cuffs having a plurality of electrodes. FIG. 10 shows one embodiment of a cuff 202. A plurality of electrodes 206 are positioned around a circumference 204 of the cuff 202. Electrical properties at a desired location may be measured by operating a select configuration or combination of electrodes 206. In the embodiment shown in FIG. 11, a cuff 210 includes a mobile gantry 216. A plurality of electrodes 214 are arranged on the mobile gantry 216 at points along a circumference 212 of the cuff 210. Electrical properties at a desired location may be measured by rotating the mobile gantry 216 to the desired location prior to operating the electrodes 214. The mobile gantry 216 rotates in a circumferential direction around the cuff 212 as indicated by arrows 218. In the embodiment shown in FIG. 12, a cuff 220 includes a mobile gantry 226. A plurality of electrodes 224 are arranged on the mobile gantry 226 along a circumference 222 of the cuff 220. Electrical properties at a desired location may be measured by translating the mobile gantry 226 to the desired location prior to operating the electrodes 224. The mobile gantry 226 translates in a longitudinal direction along a length 227 of the cuff 220 as indicated by arrow 228. FIG. 13 shows an exemplary femoral prosthesis, i.e., the femoral stem of a hip replacement prosthesis, and a cuff positioned around the joint in accordance with an embodiment. As mentioned, the external device or cuff may be placed on or around the region 108 (e.g., a joint) of the subject having the implant apparatus 102. In FIG. 13, a thigh 232 of the subject is shown including the femur bone 234. A femoral prosthesis 236 is implanted within the femur bone 234. A cuff 230, for example, cuff 202, 210 or 220 described above, may be placed around the joint or femur bone 234 by positioning the cuff 230 around the thigh 232.

Referring to FIG. 1, the plurality of electrodes 112 are coupled to the current source 114, a data acquisition module 116 and the controller 118. The electrodes 112 may be coupled to the current source 114 and the data acquisition controller 116 using a wired connection. In another embodiment, the current source may be coupled to the electrodes with a wireless connection. Current source 114 is configured to apply small, alternating currents to some or all of the electrodes 112. The resulting equipotentials are recorded (or measured) by the other electrodes 112 and the signal measured by the electrodes 112 are provide to the data acquisition module 116. This process is repeated for different configurations (or combinations) of electrodes 112. Controller 118 may be used to control the current source to apply the alternating currents for different configurations (or combinations) of electrodes 112. The data acquisition module 116 provides the measured signals to the controller 118. Controller 118 may then use the measured signals to determine electrical properties that are, for example, indicative of loosening events or conditions. In one embodiment, controller 118 generates a two-dimensional image (or tomogram) based on the measured signals using known reconstruction methods. The generated image may represent an electrical property of the region 108 or implant apparatus 102. In an embodiment, the generated image represents the distribution of conductivity in the region 108 of the subject and/or the implant apparatus 102. In another embodiment, controller 118 determines changes in the electrical properties from the measured signals, for example, changes in raw voltage data may be used to infer changes in electrical properties. The determined electrical properties or generated image may be displayed on a display 120 coupled to the controller 118. In an embodiment, the controller 118 is coupled to the display 120 with a wired connection. In another embodiment, the controller 118 is coupled to the display 120 with a wireless connection. For example, the display 120 may be a mobile device such as a tablet or phone. EIT system 104 may also include a user interface 122 coupled to the controller 118. User interface 122 may be configured to receive inputs (e.g., parameters, start data collection, change collection frequency, etc.) from an operator of the EIT system 104. User interface 122 may include, for example, a keyboard, touch screen, computer mouse, a mobile device, or other devices configured to receive information from an operator of the EIT system 104. The user interface 122 may be coupled to the controller 118 using a wired or wireless connection.

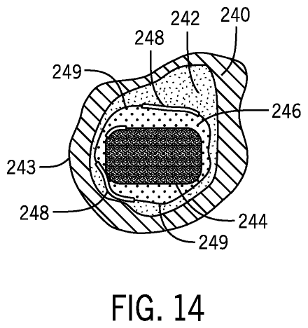

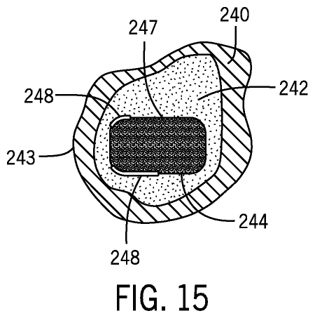

The measured electrical property (or properties) or measured changes in the electrical property (or properties) may be used to determine the loading environment and/or conditions at or near the fixations interfaces (e.g., stress/strain, crack formation, crack growth, implant motion or deformation, etc.). In one embodiment, measurements from the region 108 obtained by the EIT system 104 may be used to determine the presence and extent of connective and/or osseous tissue at or near the fixation interfaces of the implant apparatus 102. Different tissues of the human body have different electrical properties. With progressive loosening of an implant 102, the osseous tissue immediately adjacent to the prosthesis may be replaced by connective tissue. FIG. 14 shows a transverse plane cross section of an exemplary femoral prosthesis implant apparatus fixed to a bone with bone cement with a portion of a cement-bone interface occupied by connective tissue in accordance with an embodiment. FIG. 15 shows a transverse plane cross section of an exemplary femoral prosthesis implant apparatus fixed to a bone without the use of bone cement with a portion of a bone-implant interface occupied by connective tissue in accordance with an embodiment. In FIGS. 14 and 15, the cortical bone 240 and the trabecular bone 242 of a femur bone 243 are shown. A femoral prosthesis or implant 244 is positioned within the femur bone 243. In FIG. 14, bone cement 246 is disposed around the implant 244 to fix the implant 244 to the femur bone 243. Connective tissue 248 is shown located in a portion of a cement-bone interface 249. In FIG. 15, the implant 244 is a cementless implant. Connective tissue 248 is shown located in a portion of a bone-implant interface 247. Since connective tissue does not provide fixation stability to the same degree as osseous tissue, the implant 244 may be at risk of loosening. Therefore, it would be advantageous to use the EIT measurements to determine the presence and extent of connective and/or osseous tissue at or near the fixation interface. The EIT system may be designed to achieve a high signal-to-noise ratio in an appropriate range of conductivity so as to facilitate differentiation between bone and connective tissue. By repeating the EIT measurements over a period of time, changes in extent of osseous tissue can be determined to aid a clinician in determining stability of implant fixation.

In another embodiment, the EIT system 104 (shown in FIG. 1) may be used to measure the electrical properties and changes in electrical properties of a composite bone cement. As described above with respect to FIG. 5, the implant apparatus 102 (shown in FIG. 1) may include bone cement that is formed as a composite containing conductive fillers which give rise to piezoresistive behavior. Accordingly, the electrical properties of the composite bone cement, such as resistance, conductivity, etc., change in response to loading environment and/or conditions at or near the fixations interfaces (e.g., stress/strain, deformation, cracks, etc.) experienced by the bone cement. The measurements of electrical properties and images generated by the EIT system 104 may be used to evaluate the conditions at or near the cement-bone interface or the cement-implant interface, based on a known relationship between such conditions and the EIT measurements. By repeating the EIT measurements over a period of time, changes in such conditions can be determined to aid a clinician in determining stability of implant fixation. In one embodiment, the electrical property measurements and generated images may be used to detect a crack in the bone-cement. In another embodiment, the electrical property measurements and generated images may be used to detect implant migration (or displacement of the implant) within the bone cement surrounding the implant. In other embodiments, the electrical property measurements and generated images may be used to, for example, monitor the load experienced by the bone cement, detect a failure in the bone cement, or predict failures in the bone cement.

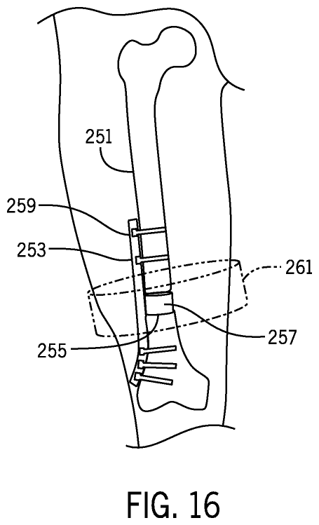

In another embodiment, the EIT system 104 (shown in FIG. 1) may be used to measure the electrical properties and/or changes in electrical properties of the composite implant. As described above with respect to FIGS. 6 and 7, the implant apparatus 102 may include an implant or a portion of the implant such as, for example, a bone interfacing portion of the implant, which is formed from a composite containing conductive fillers which give rise to piezoresistive behavior. The electrical property measurements and images generated by the EIT system 104 may be used to evaluate the conditions at or near the bone-implant or cement-implant interfaces, based on a known relationship between such conditions and EIT measurements. By repeating the EIT measurements over a period of time, changes in such conditions can be determined to aid a clinician in determining, for example, stability of implant fixation. In another embodiment, the composite implant is an orthopedic device used to fix bone fractures. FIG. 16 shows an exemplary implant apparatus used to fix bone fractures and a cuff positioned around the joint in accordance with an embodiment. In FIG. 16, a femur bone 251 is shown with a fracture (or fracture defect) 255. The fracture 255 is stabilized with screws 253 and plate 259. In addition, the fracture 256 is filled with a mesh cage 257 that is formed from piezoresistive material. An external device in the form of a cuff 261 with a plurality of electrodes (not shown) is positioned around the joint and used to measure electrical properties or changes in electrical properties within the joint including the piezoresistive mesh cage and fixation interfaces as described above. As a fracture heals, the loading environment experienced by the implant can change. Therefore, the measured electrical properties of the composite implant may be used to evaluate the progression of bone remodeling and healing in the vicinity of the composite implant. It may also be desirable to know the stability and stiffness provided by a fracture construct to optimize fracture healing. A construct that allows either too little or too much micro-motion can inhibit callus and new bone formation.

In another embodiment, the EIT system 104 (shown in FIG. 1) may be used to measure the electrical properties and/or changes in electrical properties of the implant coating. As described above with respect to FIGS. 8 and 9, the implant apparatus 102 may include an implant or a portion of the implant such as, for example, a bone interfacing portion of the implant, which is coated with a piezoresistive material. The electrical property measurements and images generated by the EIT system 104 may be used to evaluate the conditions at or near the bone-implant or cement-implant interfaces, based on known relationship between such conditions and EIT measurements.

More specific examples will be explained in the following embodiments.

### Example 1

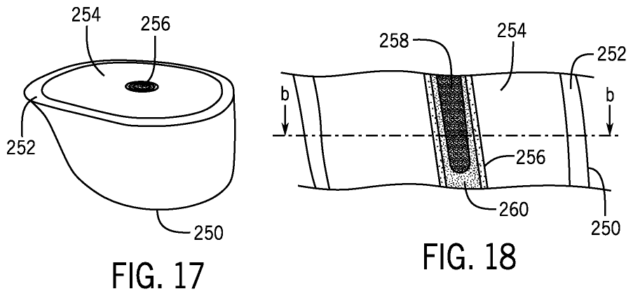

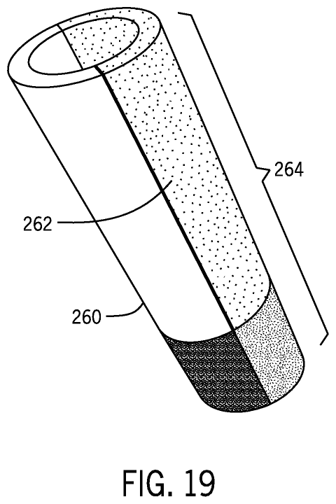

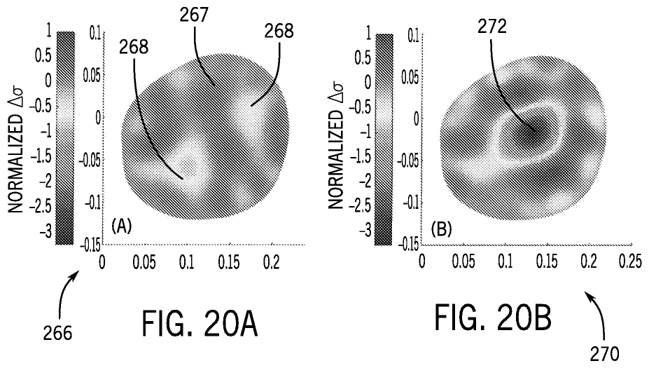

In one example, a computer simulation was conducted to demonstrate the feasibility of the disclosed system and method for measuring the electrical properties and changes in electrical properties of a composite bone cement. In particular, the simulation was conducted to determine whether the presence of a crack in the cement volume surrounding an implant could be detected using the disclosed system and method. FIG. 17 shows an example computational model of the human thigh. In FIG. 17, a portion of a thigh 250 was modeled including fatty tissue 252 and muscle 254 surrounding the native femur bone 256. The skin was modeled as a thin exterior surface layer and therefore is not shown in FIG. 17. FIG. 18 shows an example cross section computational model of a femoral prosthesis within a femur bone in accordance with an embodiment. In FIG. 18, a femoral prosthesis 258 is fixed within the femur bone 256 using a composite bone cement 260. In this example, the femoral prosthesis 258 was modeled as being formed with metal. The bone-cement 260 was modeled as being a composite containing 4% by weight of conductive nano-fillers with a baseline electrical conductivity of 5×10⁻⁸ S/cm. The known electrical conductivity values for skin, fatty tissue 252, muscle 254, femur bone 256, and the metal femoral prosthesis 268 were used in the simulation. An EIT system was used to measure the electrical conductivity within the joint both in the absence and presence of a longitudinal crack in the bone-cement volume. FIG. 19 shows an example computational model of the bone cement 260 of FIG. 18 with a simulated crack 262 along a length 264 of the bone cement 260. FIGS. 20A and 20B show the results of computer simulation. In FIGS. 20A and 20B, the normalized Δσ is the change in electrical conductivity relative to a baseline. FIG. 20A shows an example EIT image of the cross section along line b-b in FIG. 18 in the absence of a crack in the bone cement. In the absence of any cracks in the bone-cement, the EIT image 266 has a relatively uniform portion 267 in a first color (e.g., orange) with a few patches 268 in a second color (e.g., yellow) resulting from artificially introduced noise. FIG. 20B shows an example EIT image of the cross section along line b-b in FIG. 18 in the presence of a longitudinal crack in the bone cement as shown in FIG. 19. In the presence of a longitudinal crack in the bone cement, the EIT image 270 shows a distinct non-uniform color pattern. In particular, a first region 272 (e.g., dark blue) in the center of the image 270 results from the loss of conductive pathway in the bone-cement due to presence of the crack. Accordingly, the EIT measurements of the composite bone cement may be used to detect presence of cracks in the bone cement.

### Example 2

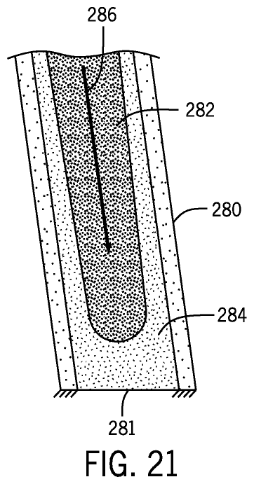

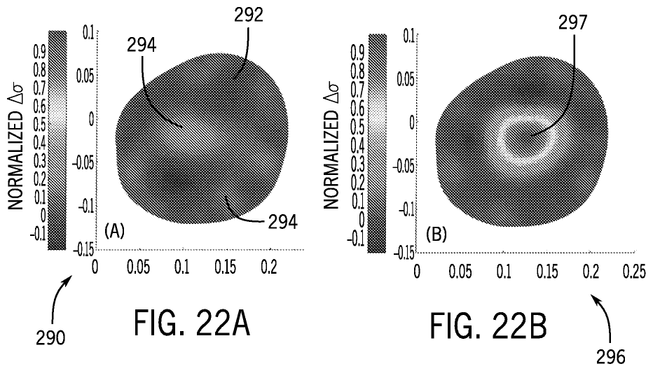

In another example, a computer simulation was conducted to demonstrate the feasibility of the disclosed system and method for measuring the electrical properties and changes in electrical properties of a composite bone cement. In particular, the simulation was conducted to determine whether displacement of an implant within the cement volume surrounding it could be detected using the disclosed system and method. FIG. 17 shows an example computational model of the human thigh. In FIG. 17, a portion of a thigh 250 was modeled including fatty tissue 252 and muscle 254 surrounding the native femur bone 256. The skin was modeled as a thin exterior surface layer and therefore is not shown in FIG. 17. FIG. 18 shows an example cross section computational model of a femoral prosthesis within a femur bone in accordance with an embodiment. In this example, the femoral prosthesis 258 was modeled as being formed with metal. The bone-cement 260 was modeled as being a composite containing 4% by weight of conductive nano-fillers with a baseline electrical conductivity of 5×10⁻⁸ S/cm. The known electrical conductivity values for skin, fatty tissue 252, muscle 254, femur bone 256, and the metal femoral prosthesis 268 were used in the simulation. An EIT system was used to measure the electrical conductivity within the joint both with and without displacement of the femoral prosthesis. FIG. 21 shows an example computational model of the femoral prosthesis of FIG. 18 with an axial displacement of the femoral prosthesis within the joint. In this example, the femur bone 280 is fixed rigidly at its bottom face 281 and a composite bone cement 284 with conductive fillers is located around the femoral prosthesis 282. An axial displacement 286 of 0.1 mm was applied to the femoral prosthesis 282 along its length 288 to simulate migration of prosthesis within the joint. FIGS. 22A and 22B show the results of computer simulation. In FIGS. 22A and 22B, the normalized Δσ is the change in electrical conductivity relative to a baseline. FIG. 22A shows an example EIT image of the cross section along line b-b in FIG. 18 in the absence of displacement of the femoral prosthesis. In the absence of any displacement of the prosthesis, the EIT image 290 has a relatively uniform region 290 in a first color (e.g., blue) with patches 294 in a second color (e.g., light blue) resulting from artificially introduced noise. FIG. 22B shows an example EIT image of the cross section along line b-b in FIG. 18 in the presence of an axial displacement of the femoral prosthesis as shown in FIG. 21. In the presence of the 0.1 mm displacement of the femoral prosthesis (shown in FIG. 21), the EIT image 296 showed a distinct non-uniform color pattern. In particular, a first region 297 (e.g., dark red) in the center of the image 296 results from the piezoresistive property of the bone cement, wherein the electrical resistivity of the bone cement decreases due to the increased stresses/strains in the bone cement caused by the displacement of the femoral prosthesis.

### Example 3

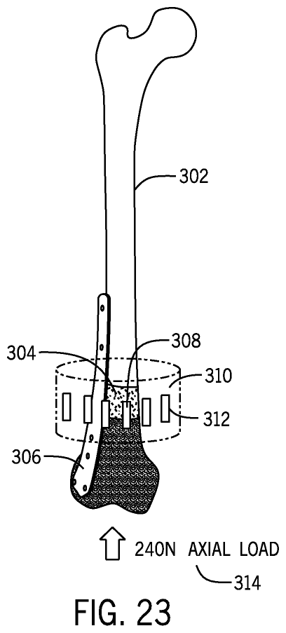

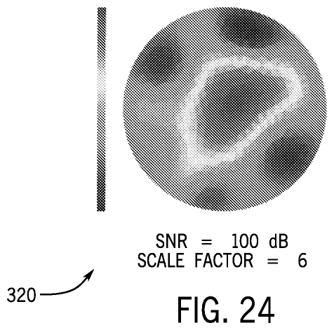

In another example, a computer simulation was conducted to evaluate the feasibility of using an EIT system to detect implant-bone load sharing. As discussed above with respect to FIG. 16, a composite implant may be an orthopedic device to fix fractures. FIG. 23 shows an example computational model of an implant apparatus used to fix bone fractures and a cuff positioned around the joint. In FIG. 23, a femur bone 302 model with a 30 mm segmental defect (fracture) 304 is shown. An angular locking plate 306 with screws (nor shown) was used to stabilize the fracture 304. In this example, the fracture 304 was filled with a soft-segment (0.5% stiffness of native bone) representing a piezoresistive cage 308 made of a composite with baseline electrical conductivity of 2×10⁻⁵ S/m. A 240N axial load was applied to the distal end of the femur bone 302 and the change in electrical conductivity within the fracture 304 region was calculated. A cuff 310 with a plurality of electrodes 312 may be positioned around the joint and used to measure electrical properties or changes in electrical properties within the joint including the implant apparatus and fixation interfaces as described above. FIG. 24 shows an example EIT image showing a change in conductivity within the fracture shown in FIG. 23 relative to an unloaded condition. The EIT image 320 shows a notable change in conductivity resulting from piezoresistive response of the cage under applied load is clearly detectable above the artificially introduced noise (signal to noise ration 100 dB). This illustrates that the disclosed EIT system and method may be used to detect implant-bone load sharing.

### Example 4

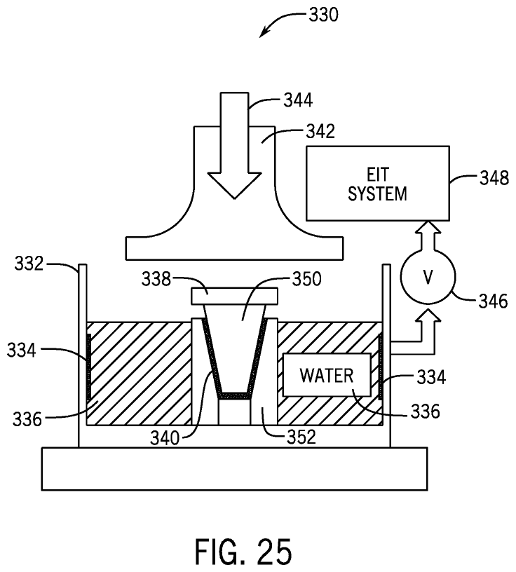

In this example, an experimental apparatus and setup were used to evaluate system and methods described herein for determining conditions within or near an implant apparatus in a subject. In particular, the experimental setup was used to evaluate the disclosed methods with regard to detecting and monitoring load induced deformation and fractures of a piezoresistive bone cement in a simulated implant apparatus. FIG. 25 shows an example experimental setup including an experimental test phantom. In the example setup of FIG. 25, the experimental setup 330 includes a phantom tank 332 measuring 150 mm in diameter and 80 mm deep that was filled with deionized water 336. Sixteen (16) electrodes 334 were positioned on an internal surface of the tank. To reduce oxidation, the electrodes 334 were coated with silver paste. A conical specimen or sample 338 was used to simulate a prosthesis (e.g., a hip implanted and cemented into a cavity of a femur bone) and act as a prosthetic surrogate. The conical specimen 338 is configured to simulate an implant-cement-bone interaction. In one embodiment, the conical specimen 338 was configured to apply compressive and shear forces to a layer of bone cement 340 that is positioned between a first section 350 and a second section 352 of the conical specimen 338. Holes (not shown) were provided in the second region 352 of the conical specimen 338 to expose the bone cement 340 to the water 336 in the phantom tank 332 to simulate a physiologic fluid environment. The bone cement 340 was formed by adding low level fraction of carbon fiber (CF) (i.e., micro-scale chopped carbon fibers that are 3 mm in length and 7 μm in diameter) to PMMA to create a piezoresistive bone cement. In this example, the amount of CF in the bone cement 340 is above the percolation threshold (i.e., the lower limit of CF needed to form a well-connected network within the PMMA through which electrical current can propagate). The phantom tank 332 is configured so that an MTS load frame 342 may be used to apply a load (indicated by arrow 344) to the conical specimen 338 as EIT measurements are taken. Electrodes 334 are part of an EIT system 348 used to measure electrical properties of the conical specimen 338. In this example, the EIT system 348 includes a power source, a linear regulator and an adjustable electrical resistor that were used to inject 25 μA DC currents via the electrodes 334. The resulting voltages 346 were measured via an Arduino mega data acquisition board.

In this example, EIT system 348 was used to monitor deformation and damage-induced conductivity changes of the bone cement 340 interfacial layer. The EIT system 348 estimates the conductivity distribution of a domain from a series of non-invasive current-voltage measurements collected at the domain boundary. EIT system 348 minimizes the difference between a set of experimentally measured boundary voltages and another set of computationally predicted boundary voltages. The process of computationally predicting voltages is referred to as the forward problem and begins with Laplace's equation for steady-state diffusion in the absence of internal current sources as shown in Eq. (1).

∇·σ∇ϕ=0 (1)

In the preceding, σ is the conductivity distribution of the domain and ϕ is the domain potential. The complete electrode model boundary conditions are then enforced on Eq. (1) as shown in Eqs. (2) and (3). Equation (2) accounts for a voltage drop between the domain and the assumed to be perfectly conducting electrodes due to contact impedance. Equation (3) enforces conservation of charge. In these equations, zₗ is the contact impedance between the lth electrode and the domain, n is an outward pointing normal vector, Vₗ is the voltage on the lth electrode, Eₗ is the length of the lth electrode, and L is the total number of electrodes.

ϕ+zₗσ∇ϕ·n=Vₗ (2)

Σₗ₌₁^{L}∫_{E}_{ₗ}σ∇ϕ·ndSₗ=0 (3)

Equations (1)-(3) are solved via the finite element method as shown in Eq. (4). In Eq. (4), A_{M} is the standard finite element stiffness matrix for steady-state diffusion, Φ is a vector of domain potentials, V is a vector of electrode voltages, and I is a vector of currents injected at the electrodes. A_{Z}, A_{w}, and A_{D} are formed as respectively shown in Eqs. (5), (6), and (7) where wᵢ is the ith finite element interpolation function. Two-dimensional triangles with linear interpolation functions are used.

$$\begin{matrix}[ \begin{matrix}{A}_{M} + {A}_{Z} & {A}_{W} \\ {A}_{W}^{T} & {A}_{D}\end{matrix} ] [ \begin{matrix}Φ \\ V\end{matrix} ] = [ \begin{matrix}0 \\ I\end{matrix} ] & ( 4 ) \\ {A}_{Z ij} = {\sum }_{l = 1}^{L} {\int }_{{E}_{l}} \frac{1}{{z}_{l}} {w}_{i} {w}_{j} {dS}_{l} & ( 5 ) \\ {A}_{W li} = - {\int }_{{E}_{l}} \frac{1}{{z}_{l}} {w}_{i} {dS}_{l} & ( 6 ) \\ {A}_{D} = diag ( \frac{{E}_{l}}{{z}_{l}} ) & ( 7 )\end{matrix}$$

The EIT inverse problem is the process of recovering the conductivity distribution. Herein, a one-step linearization process is employed in which experimental voltages are collected at some initial time, again at some later time (i.e. after some conductivity-changing event), and the conductivity change between these two times is sought. This method is used because it is more robust to noise and errors arising from factors such as discrepancies between electrode placement in the experiment and the model than absolute conductivity imaging algorithms. Mathematically, a value of Δσ is sought to minimize the difference between δV=V(t₂)−V(t₁) and W=F(σ₀+Δσ)−F(σ₀) in the least-squares sense as shown in Eq. (8).

$${Δσ}^{*} = {arg min}_{Δσ} \frac{1}{2} ( {‖ W ( Δσ ) - δ V ‖}_{2}^{2} ) \qquad ( 8 )$$

In the preceding, δV is the difference between experimentally collected voltages taken at times tᵢ and t₂, F(⋅) is a vector of electrode voltages predicted by the previously described forward problem for the conductivity distribution provided in its argument, σ₀ is an estimate of the background conductivity, Δσ is the conductivity change between times t₁ and t₂, and Δσ* is a conductivity change that satisfies the minimization. Note that σ has been boldfaced in anticipation of discretization by the finite element method. To solve this minimization, F(σ₀+Δσ) is approximated by using a Taylor series expansion and retaining only the linear terms as shown in Eq. (9).

$${Δσ}^{*} = {min}_{Δσ \leq 0} \frac{1}{2} ( {‖ J Δσ - δ V ‖}_{2}^{2} + α {‖ L Δσ ‖}_{2}^{2} ) \qquad ( 10 )$$

By substituting Eq. (9) into W and defining the sensitivity matrix as J=∂F(σ₀)/∂σ, Eq. (8) can be recast as shown in Eq. (10).

$$F ( {σ}_{0} - Δσ ) ≅ F ( {σ}_{0} ) + \frac{\partial F ( {σ}_{0} )}{\partial σ} Δσ \qquad ( 9 )$$

Beyond eliminating W, two important differences between Eqs. (8) and (10) should be noted. First, a constraint has been added that the conductivity change be less than or equal to zero. This constraint was physically motivated by the fact that the CF/PMMA bone cement 340 exhibited a conductivity loss with compression. And second, a regularization term, L, has been added. This regularization is necessary to recover a physically meaningful solution as the EIT inverse problem is under determined and ill posed. Here, L is the discrete Laplace operator and a is a scalar hyper parameter used to control the amount of regularization.

In this example, three conically shaped prosthetic surrogate specimens 338 with 1.0, 1.5, and 2.0 vol. % CF cement layers 340 were tested. Each specimen was incrementally loaded up to 4000 N. EIT measurements were collected at each load as the load was held constant. After loading up to 4000 N, each specimen was loaded until failure (occurring around 5700, 6700 and 5700 N for 1.0, 1.5 and 2.0 vol. % CF samples, respectively). EIT measurements were again collected after each specimen failed.

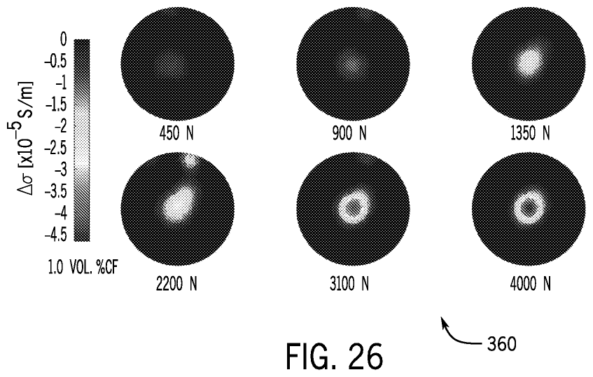

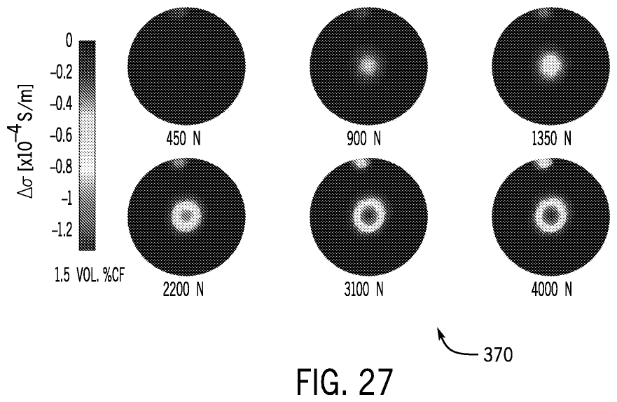

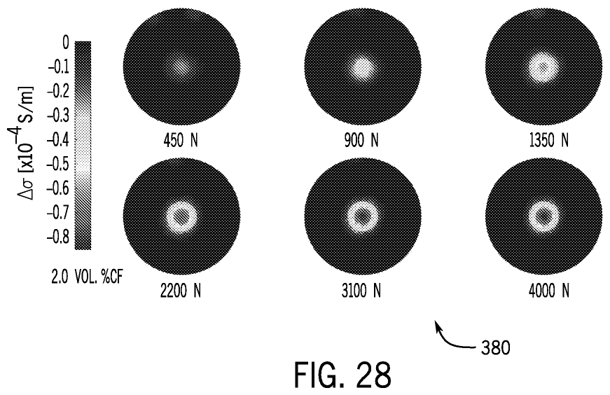

As mentioned, the experimental setup 330 was used to evaluate the disclosed methods with regard to load monitoring. In the load monitoring example, EIT images for 1.0, 1.5 and 2.0 vol. % CF cement layers 340 were obtained and are shown in FIGS. 26, 27 and 28, respectively. FIG. 26 shows example EIT images 360 illustrating conductivity changes of a bone cement with 1.0 vol. % carbon fiber as a function of applied load. FIG. 27 shows example EIT images 370 illustrating conductivity changes of a bone cement with 1.5 vol. % carbon fiber as a function of applied load. FIG. 28 shows example EIT images 380 illustrating conductivity changes of a bone cement with 2.0 vol. % carbon fiber as a function of applied load. In these images 360, 370, 380, EIT measurements at a load of 50 N were used as a baseline for difference imaging. This low level of loading was used as a baseline rather than a load-free condition because it is more representative of a person at rest (e.g., sitting or lying). EIT images corresponding to 450, 900, 1350 N, or 2200 N may be representative of a range of quasi-static loads incurred by normal, low-stress activities such as walking or climbing stairs. Large forces (>2200 N) can be interpreted as being representative of dynamic loads incurred during more strenuous activities such as impact loads during running, jumping, or other forms of exercise. Consideration of these larger loads is important because, as mentioned in the introduction, the most at-risk population for aseptic implant loosening are those who lead more active lifestyles.

Several observations may be made from the images 360, 370, 380 shown in FIGS. 26, 27 and 28. First, for each CF volume fraction considered, the EIT images identify the prosthetic location in the center of the phantom tank 332 (shown in FIG. 25). Second, beyond mere location identification, the EIT images distinguish between increasing loads. That is, as the load magnitude increases, the conductivity change at the center of the phantom tank intensifies. This is important because it means that the disclosed combination of composite piezoresistive bone cement and EIT may be utilized for in vivo load monitoring. This may be useful to understanding and mitigating the factors which precipitate aseptic loosening. And third, the EIT images demonstrate that even relatively modest loads (i.e., <900 N) can be detected.

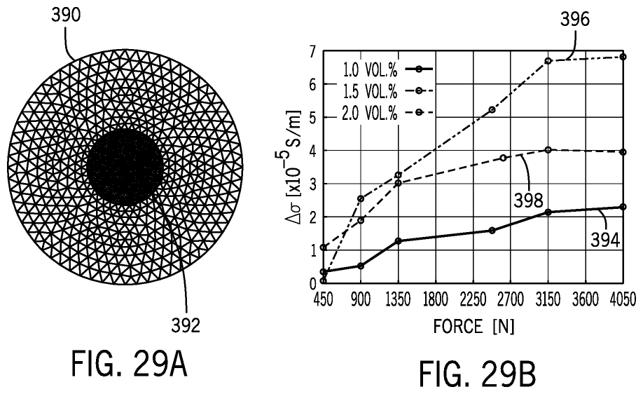

To further demonstrate the capability of this method for load distinguishability, the average conductivity change as predicted by EIT in the center of the phantom where the prosthetic surrogate is located as a function of applied load was calculated. This is shown in FIG. 29A and FIG. 29B where there is a clear trend of increasing conductivity change magnitude with increasing load. FIG. 29A illustrates an example finite element mesh used for EIT reconstructions. The average conductivity change of elements in a first region 392 corresponding to the implant location is plotted as a function of the applied force. FIG. 29B is an example graph showing average magnitude of conductivity change as imaged by EIT versus applied force. Curve 394 represents the average magnitude of conductivity change for bone cement with 1.0 vol. % CF, curve 396 represents the average magnitude of conductivity change for bone cement with 1.5 vol. % CF, and curve 398 represents the average magnitude of conductivity change for bone cement with 2.0 vol. % CF. There is a clear correspondence between applied force and conductivity change thereby demonstrating the combination of composite piezoresistive bone cement and EIT may be utilized for load monitoring.

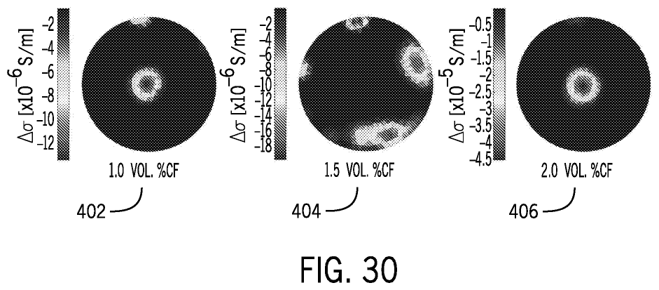

As mentioned, the experimental setup 330 (shown in FIG. 25) was also used to evaluate the disclosed methods with regard to failure detection. That is, in this example each prosthetic surrogate (1.0 vol. % CF, 1.5 vol. % CF, 2.0 vol. % CF) was loaded past 4000 N until a gross failure event occurred. After failure, EIT measurements were collected. FIG. 30 shows example EIT images obtained post failure for bone cement specimens with 1.0 vol. % CF, 1.5 vol. % CF and 2.0 vol. % CF. EIT image 402 is an image for a bone cement CF volume fraction of 1.0 vol. %, image 404 is an image for a bone cement CF volume fraction of 1.5 vol. %, and image 406 is an image for a bone cement CF volume fraction of 2.0 vol. %. The 50 N case is again used as a baseline for difference imaging. Failure-induced conductivity increase are clearly seen for all specimens. It should be noted that these images 402, 404, 406 were formed with the constraint that the conductivity change be less than or equal to zero.

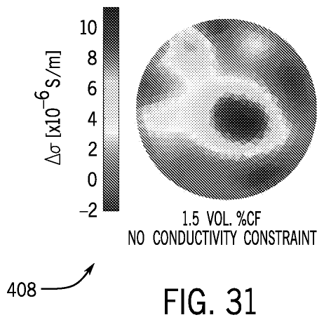

Two observations may be made from the images 402, 404 and 406 shown in FIG. 30. First, a conductivity loss due to failures can be clearly seen at the center of the phantom for both the 1.0 and 2.0 vol. % specimens. The conductivity loss is a consequence of the conductive bone cement fracturing and the ruptured volume being filled with less conductive deionized water. And second, considering the image 404 which corresponds to the 1.5 vol. % CF specimen, EIT appears to not have detected that the prosthetic has failed. To better understand this, it should be noted that the EIT problem was formulated such that conductivity changes were constrained to be less than or equal to zero. Upon removing this constraint and resolving the EIT inverse problem for the 1.5 vol. % specimens, the conductivity change shown in FIG. 31 is recovered. FIG. 31 shows an example EIT image of a 1.5 vol. % CF specimen without any constraints on the conductivity change. The image 408 shows that the EIT detects a conductivity increase due to a de-bonding failure event (as opposed to CF/PMMA fracture for the 1.0 and 2.0 vol. % specimens).

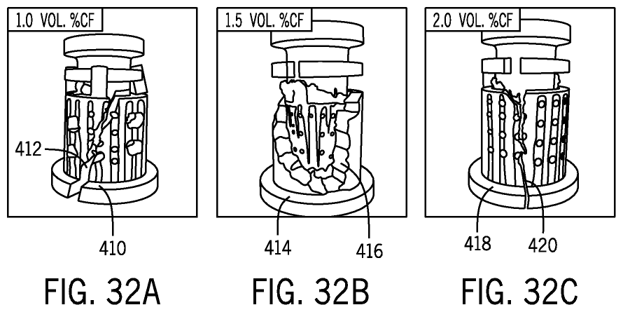

Image 408 in FIG. 31 illustrates a clear conductivity change at the center of the phantom. The conductivity change is positive indicating that the region has become more conductive due to the failure event. To understand what is happening here, the post-failure specimens as shown in FIGS. 32A, 32B and 3C are examined. FIG. 32A shows a specimen with a bone cement CF volume fraction of 1.0 vol. % CF post failure. FIG. 32B shows a specimen with a bone cement CF volume fraction of 1.5 vol. % CF post failure. FIG. 32C shows a specimen with a bone cement CF volume fraction of 2.0 vol. % CF post failure. For specimen 410 in FIG. 32A with 1.0 vol. % CF and specimen 418 in FIG. 32C with 2.0 vol. % CF, both the bone cement and implant surrogate have cracked. FIGS. 32A and 32C show a vertical crack 412, 420 through both cement and the implant surrogate. However, for the specimen 414 in FIG. 32B with 1.5 vol. % CF, only the implant surrogate has broken 416. This significantly increased the surface area of CF/PMMA bone cement exposed to the water. Additionally, the bone cement did not actually break. Because the CF/PMMA is more conductive than the deionized water and a greater amount of it is in direct contact with the water after the failure event, there is an easier path for electric current to flow and hence an overall higher apparent conductivity in the center of the phantom tank just as predicted by EIT. The failure shown in FIG. 32B for the specimen with 1.5 vol. % CF may be thought of as being similar to de-bonding between PMMA and bone or between PMMA and implant. This particular failure mode of the 1.5 vol. % indicates that EIT data and images may be used to distinguish between failure types. That is, a fracture or breakage of the cement will result in a conductivity loss whereas a de-bonding event increases EIT-imaged conductivity.

This example shows that EIT may be used to identify loading on implants and bone cement, distinguish between increasing load magnitudes, detect failure (i.e., cement cracking and cement de-bonding), and distinguish between failure modes. As mentioned above, in this example polymethyl methacrylate (PMMA) bone cement was made conductive by modification with low weight fractions conductive fibers above the percolation threshold (i.e., the lower limit of CF needed to form a well-connected network within the PMMA through which electrical current can propagate). Mechanical effects such as deformation or fracture alter the connectedness of the CF network and consequently manifest as a conductivity change. In other words, the material is piezoresistive. Therefore, conductivity changes can be used to monitor load transfer across the bone cement as a precursor to loss of implant fixation or outright failure of the bone cement.

EIT was used to monitor conductivity changes of the CF-modified PMMA bone cement. EIT has several advantages compared to more common modalities such as radiographs. First, unlike ionizing radiation, low levels of electrical current are physiologically benign. Second, EIT is very low cast, generally only requiring precision current supplies, voltmeters, and modest computational power. And third, EIT has high temporal resolution. Optimized EIT systems may generate images in nearly real time. In an embodiment, an EIT system may be used continuously (e.g., via a wearable cuff with built-in electrodes) on, for example, high-risk patients to provide real-time information on in vivo implant fixation, load transfer, and failure.

Computer-executable instructions for determining conditions within or near an implant or fixation interfaces in a subject according to the above-described methods may be stored on a form of computer readable media. Computer readable media includes volatile and nonvolatile, removable, and non-removable media implemented in any method or technology for storage of information such as computer readable instructions, data structures, program modules or other data. Computer readable media includes, but is not limited to, random access memory (RAM), read-only memory (ROM), electrically erasable programmable ROM (EEPROM), flash memory or other memory technology, compact disk ROM (CD-ROM), digital volatile disks (DVD) or other optical storage, magnetic cassettes, magnetic tape, magnetic disk storage or other magnetic storage devices, or any other medium which can be used to store the desired instructions and which may be accessed by a system (e.g., a computer), including by internet or other computer network form of access.

The present invention has been described in terms of one or more preferred embodiments, and it should be appreciated that many equivalents, alternatives, variations, and modifications, aside from those expressly states, are possible and within the scope of the invention.

1. A system for determining conditions within or near an implant apparatus in region of a subject, the system comprising:
an implant apparatus configured to be piezoresistive, the implant apparatus positioned in a region of a subject having a surface;
an electrical impedance tomography system comprising:
a plurality of electrodes disposed on the surface of the region of the subject, each of the plurality of electrodes configured to apply a current to the region of the subject and to receive measurement signals from the region of the subject in response to an applied current;
a current source coupled to the plurality of electrodes and configured to provide a current to at least one of the plurality of electrodes;
a controller coupled to the plurality of electrodes and the current source, the controller configured to control the current source to provide a current to at least one of the plurality of electrodes, to receive the measurement signals from at least one of the plurality of electrodes, and to determine at least one electrical property associated with the implant apparatus based on the measurement signals; and
a display coupled to the controller and configured to display the at least one electrical property.

2. The system according to claim 1, wherein the implant apparatus comprises:
a surface; and
bone cement disposed around the surface of the implant apparatus, wherein the bone cement is composed of a composite material having conductive fillers configured to provide piezoresistive behavior.

3. The system according to claim 1, wherein the implant apparatus comprises an implant composed of a composite material having conductive fillers configured to provide piezoresistive behavior.

4. The system according to claim 3, wherein the conductive fillers are disposed within the implant near an implant-bone fixation interface.

5. The system according to claim 3, wherein the implant apparatus further comprises bone cement and the conductive fillers are disposed within the implant near an implant-cement interface.

6. The system according to claim 1, wherein the implant apparatus comprises:
a surface; and
a piezoresistive material disposed on the surface of the implant apparatus.

7. The system according to claim 1, wherein the electrical property is one of impedance, conductivity, permittivity, or resistivity.

8. The system according to claim 1, wherein the region of the subject is a joint.

9. The system according to claim 8, wherein the implant apparatus is a joint prosthesis.

10. The system according to claim 1, wherein the plurality of electrodes are positioned on an external device that is configured to be placed around the region of the subject.

11. The system according to claim 10, wherein the external device is a cuff.

12. A method for determining conditions within or near an implant apparatus in region of a subject, the method comprising:
providing a plurality of electrodes on the surface of the region of the subject, the region of the subject having a surface and including an implant apparatus configured to be piezoresistive;
applying a current to the region of the subject with at least one of the plurality of electrodes;
detecting measurement signals with at least one of the plurality of electrodes, the measurement signals generated in response to the applied current;
determining at least one electrical property associated with the implant apparatus based on the measurement signals; and
displaying the at least one electrical property on a display.

13. The method according to claim 12, wherein the implant apparatus comprises:
a surface; and
bone cement disposed around the surface of the implant apparatus, wherein the bone cement is composed of a composite material having conductive fillers configured to provide piezoresistive behavior.

14. The method according to claim 12, wherein the implant apparatus comprises an implant composed of a composite material having conductive fillers configured to provide piezoresistive behavior.

15. The method according to claim 14, wherein the conductive fillers are disposed within the implant near an implant-bone fixation interface.

16. The method according to claim 14, wherein the implant apparatus further comprises bone cement and the conductive fillers are disposed within the implant near an implant-cement interface.

17. The method according to claim 12, wherein the implant apparatus comprises:
a surface; and
a piezoresistive material disposed on the surface of the implant apparatus.

18. The method according to claim 12, wherein the electrical property is one of impedance, conductivity, permittivity, or resistivity.

19. The method according to claim 12, wherein the region of the subject is a joint.

20. The method according to claim 19, wherein the implant apparatus is a joint prosthesis.
